(19) United States
(12) Patent Application Publication
(10) Pub. No.: US 2024/0268724 A1
(43) Pub. Date: Aug. 15, 2024

(54) SYSTEM AND METHOD FOR REDUCING CGM WARM-UP TIME BY APPLICATION OF OPTICAL ENERGY

(71) Applicant: Insulet Corporation, Acton, MA (US)

(72) Inventor: James CAUSEY, Simi Valley, CA (US)

(21) Appl. No.: 18/437,595

(22) Filed: Feb. 9, 2024

Related U.S. Application Data
(60) Provisional application No. 63/484,626, filed on Feb. 13, 2023.

(51) Int. Cl. A61B 5/1491; A61B 5/00; A61B 5/145; A61B 5/1455

(52) U.S. Cl. CPC A61B 5/1491; A61B 5/14532; A61B 5/1455; A61B 5/4836; A61B 2562/0233

(57) ABSTRACT

Disclosed herein is a system and method providing an improvement to a medical device such as a wearable CGM as part of an automated drug delivery system. The improvement provides optical energy to the wound site caused by the insertion of a sensing element into the skin of the user to hasten the healing of the wound, resulting in an improvement in the accuracy of readings for the CGM in a timelier manner. This tends to lessen the effect of the warm-up period of the CGM and improves the response of the automated drug delivery system to better manage the user's blood glucose levels.

### CROSS-REFERENCE TO RELATED APPLICATION

This application claims priority to and the benefit of U.S. Provisional Application No. 63/484,626, filed Feb. 13, 2023, the entirety of which is incorporated herein by reference.

### BACKGROUND

Many conventional automated drug delivery (ADD) systems are well known. Such ADD systems typically include a drug delivery device that can be designed to deliver any type of liquid drug to a user. In specific embodiments, the drug delivery device can be a wearable drug delivery device, for example, an OmniPod® drug delivery device manufactured by Insulet Corporation of Acton, Massachusetts. The drug delivery device can be a drug delivery device such as those described in U.S. Pat. No. 7,303,549, U.S. Pat. No. 7,137,964, or U.S. Pat. No. 6,740,059.

Such drug delivery devices typically include a positive displacement pumping mechanism to force a liquid drug from a reservoir through a fluid path to the patient. The fluid path typically comprises a flexible tube or needle coupled, at one end, to the reservoir. The other end of the fluid path is coupled to a cannula which is inserted under the skin of the patient for delivery of the liquid drug. The cannula may be inserted via a needle mechanism wherein a needle/cannula combination is forced by an actuator into the skin of the user and thereafter the needle is withdrawn, leaving the cannula in place. The cannula may have one or more ports defined at or proximal a distal end thereof through which the liquid drug is dispensed.

In the case of an automated drug delivery (ADD) systems designed to deliver insulin for the management of diabetes, the system may also include a wearable continuous glucose monitor (CGM). The CGM may insert a sensor into the skin of a user using the sensor or using a needle in an arrangement similar to the insertion of the cannula by the drug delivery device, wherein the needle is withdrawn, leaving the sensor in place under the skin of the user. Insertion of the needle into the skin causes a wound which may trigger a foreign body response at the insertion site, which may include inflammation of the skin. Such inflammation may cause readings from the CGM to be inaccurate for a period of time after insertion of the sensor (i.e., the “warm-up time”). The warm-up time for a CGM sensor varies from manufacturer to manufacturer and could be high as 12 hours after insertion of the sensor. During this period, the user may experience a disturbance in the management of the user's blood glucose levels, which may result in a dangerous situation for the user. Therefore, it is desirable to reduce or minimize the warm-up time during which the readings from the CGM are less reliable.

### SUMMARY

This Summary is provided to introduce a selection of concepts in a simplified form that are further described below in the Detailed Description.

Disclosed herein is an improvement to a wearable medical device, such as a CGM. Systems and methods described herein provide optical energy to the wound site to hasten the healing of the wound. In the case of a CGM, this results in an improvement in the accuracy of readings for the CGM in a timelier manner. This tends to lessen the effect of the warm-up period of the CGM and improves the response of the automatic drug delivery system to better manage the user's blood glucose levels. In the case of a wearable drug delivery device, or an infusion port connected to a drug delivery device, applying optical energy to the wound site where a cannula or needle has been inserted to enable drug delivery under the skin similarly hastens the healing of the wound and may result in improved drug absorption under the skin (e.g., absorption of insulin when using an insulin delivery device).

In one embodiment, the application of optical energy to the skin occurs via exposing the skin to red or near-infrared light having a wavelength in the range of 630 nm-850 nm, which is known to promote wound healing and have anti-inflammatory effects. One or more light sources emitting red and infrared light in the desired wavelength range is built into the wearable medical device, such as a CGM or the drug delivery device, and programmatically modulated, typically after insertion of the sensor and/or cannula and, if using a needle to aid insertion, after retraction of the needle. In a second embodiment, thermal energy may be applied directly to the wound area via heating of the sensor and/or cannula using a resistive element. In yet a third embodiment, a combination of the first two embodiments may be used.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates a functional block diagram of an exemplary system suitable for use with the devices disclosed herein.

FIG. 2 is a schematic diagram showing application of the infrared light to the wound location.

### DETAILED DESCRIPTION

Various embodiments of the present invention include systems and methods for delivering a medication to a user using a drug delivery device, either autonomously, or in accordance with a wireless signal received from an electronic device. In various embodiments, the electronic device may be a user device comprising a smartphone, a smart watch, a smart necklace, a module attached to the drug delivery device, or any other type or sort of electronic device that may be carried by the user or worn on the body of the user and that executes an algorithm that computes the times and dosages of delivery of the medication. Alternatively, the drug delivery device operates an algorithm stored on the drug delivery device itself, without reliance on a remote electronic device for delivering medicament to the user.

For example, the user device or drug delivery device may execute an “artificial-pancreas” (AP) algorithm that computes the times and dosages of delivery of insulin. The user device and/or drug delivery device may also be in communication with a sensor, such as a glucose sensor or a continuous glucose monitor (CGM), that collects data on a physical attribute or condition of the user, such as a glucose level. The sensor may be disposed in or on the body of the user and may be part of the drug delivery device or may be a separate device.

Alternatively, the drug delivery device may be in communication with the sensor in lieu of or in addition to the communication between the sensor and the user device. The communication may be direct (if, e.g., the sensor is integrated with or otherwise a part of the drug delivery device) or remote/wireless (if, e.g., the sensor is disposed in a different housing than the drug delivery device). In these embodiments, the drug delivery device contains computing hardware (e.g., a processor, memory, firmware, etc.) that executes some or all of the algorithm that computes the times and dosages of delivery of the medication.

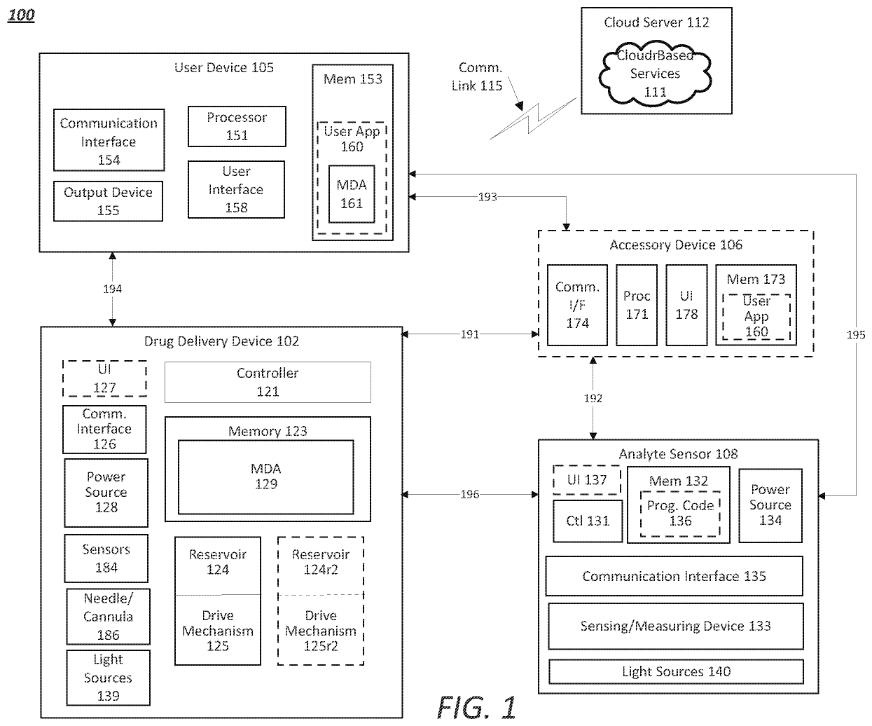

FIG. 1 illustrates a functional block diagram of an exemplary drug delivery system 100 suitable for implementing the systems and methods described herein. The drug delivery system 100 may implement (and/or provide functionality for) a medication delivery algorithm, such as an artificial pancreas (AP) application, to govern or control the automated delivery of a drug or medication, such as insulin, to a user (e.g., to maintain euglycemia—a normal level or range of glucose in the blood). The drug delivery system 100 may be an automated drug delivery system that may include a drug delivery device 102 (which may be wearable or may include an infusion set), an analyte sensor 108 (which may also be wearable), and a user device 105.

Drug delivery system 100, in an optional example, may also include an accessory device 106, such as a smartwatch, a personal assistant device, a smart insulin pen, or the like, which may communicate with the other components of system 100 via either a wired or wireless communication links 191-193.

### User Device

The user device 105 may be a computing device such as a smartphone, a smartwatch, a tablet, a personal diabetes management (PDM) device, a dedicated diabetes therapy management device, or the like. In an example, user device 105 may include a processor 151, device memory 153, a user interface 158, and a communication interface 154. The user device 105 may also contain analog and/or digital circuitry that may be implemented as a processor 151 for executing processes based on programming code stored in device memory 153, such as user application 160 incorporating medication delivery algorithm (MDA) 161 to manage a user's blood glucose levels and for controlling the delivery of the drug, medication, or therapeutic agent to the user, as well for providing other functions, such as calculating carbohydrate-compensation dosage, a correction bolus dosage and the like as discussed below. The user device 105 may be used to activate, deactivate, trigger a needle/canula insertion, program, adjust settings, and/or control operation of drug delivery device 102 and/or the analyte sensor 108 as well as the optional smart accessory device 106.

The processor 151 may also be configured to execute programming code stored in device memory 153, such as the user app 160. The user app 160 may be a computer application that is operable to deliver a drug based on information received from the analyte sensor 108, the cloud-based services 111 and/or the user device 105 or optional accessory device 106. The memory 153 may also store programming code to, for example, operate the user interface 158 (e.g., a touchscreen device, a camera or the like), the communication interface 154 and the like. The processor 151, when executing user app 160, may be configured to implement indications and notifications related to meal ingestion, blood glucose measurements, and the like. The user interface 158 may be under the control of the processor 151 and be configured to present a graphical user interface that enables the input of a meal announcement, adjust setting selections and the like as described herein.

In a specific example, when the user app 160 includes MDA 161, the processor 151 is also configured to execute a diabetes treatment plan (which may be stored in a memory) that is managed by user app 160. In addition to the functions mentioned above, when user app 160 is an AP application, it may further provide functionality to determine a carbohydrate-compensation dosage, a correction bolus dosage and determine a real-time basal dosage according to a diabetes treatment plan. In addition, as an MDA 161, user app 160 provides functionality to output signals to the drug delivery device 102 via communications interface 154 to deliver the determined bolus and/or basal dosages.

The communication interface 154 may include one or more transceivers that operate according to one or more radio-frequency protocols. In one embodiment, the transceivers may comprise a cellular transceiver and a Bluetooth® transceiver. The communication interface 154 may be configured to receive and transmit signals containing information usable by user app 160.

User device 105 may be further provided with one or more output devices 155 which may be, for example, a speaker or a vibration transducer, to provide various signals to the user.

### Drug Delivery Device

In various exemplary embodiments, drug delivery device 102 may include a reservoir 124 and drive mechanism 125, which are controllable by controller 121, executing a medication delivery algorithm (MDA) 129 stored in memory 123, which may perform some or all of the functions of the AP application described above, such that user device 105 may be unnecessary for drug delivery device 102 to carry out drug delivery and control. Alternatively, controller 121 may act to control reservoir 124 and drive mechanism 125 based on signals received from user app 160 executing on a user device 105 and communicated to drug delivery device 102 via communication link 194. Drive mechanism 125 may operate to longitudinally translate a plunger through the reservoir, so as to force the liquid drug through an outlet fluid port to needle/cannula 186. Alternatively, other types of drive mechanisms may be used.

In an alternate embodiment, drug delivery device 102 may also include an optional second reservoir 124-2 and second drive mechanism 125-2 which enables the independent delivery of two different liquid drugs. As an example, reservoir 124 may be filled with insulin, while reservoir 124-2 may be filled with glucagon, or Pramlintide, or GLP-1. In some embodiments, each of reservoirs 124, 124-2 may be configured with a separate drive mechanism 125, 125-2, respectively, which may be separately controllable by controller 121 under the direction of MDA 129. Both reservoirs 124, 124-2 may be connected to a common needle/cannula 186.

Drug delivery device 102 may be optionally configured with a user interface 127 providing a means for receiving input from the user and a means for outputting information to the user. User interface 127 may include, for example, light-emitting diodes, buttons on a housing of drug delivery device 102, a sound transducer, a micro-display, a microphone, an accelerometer for detecting motions of the device or user gestures (e.g., tapping on a housing of the device) or any other type of interface device that is configured to allow a user to enter information and/or allow drug delivery device 102 to output information for presentation to the user (e.g., alarm signals or the like).

Drug delivery device 102 includes a patient interface 186 for interfacing with the user to deliver the liquid drug. Patient interface may be, for example, a needle or cannula for delivering the drug into the body of the user (which may be done subcutaneously, intraperitoneally, or intravenously). Similar to CGM 108, drug delivery device 102 may be provided with one or more light sources 139 providing red and near-infrared light to the wound site where the needle/cannula 186 was inserted into the skin. Drug delivery device 102 may further include a mechanism for inserting the needle/cannula 186 into the body of the user, which may be integral with or attachable to drug delivery device 102. The insertion mechanism may comprise, in one embodiment, an actuator that inserts the needle/cannula 186 under the skin of the user and thereafter retracts the needle, leaving the cannula in place. The actuator may be triggered by user device 105 or may be a manual firing mechanism comprising springs or other energy storing mechanism, that causes the needle/cannula 186 to penetrate the skin of the user.

In one embodiment, drug delivery device 102 includes a communication interface 126, which may be a transceiver that operates according to one or more radio-frequency protocols, such as Bluetooth®, Wi-Fi, near-field communication, cellular, or the like. The controller 121 may, for example, communicate with user device 105 and the analyte sensor 108 via the communication interface 126.

In some embodiments, drug delivery device 102 may be provided with one or more sensors 184. The sensors 184 may include one or more of a pressure sensor, a power sensor, or the like that are communicatively coupled to the controller 121 and provide various signals. For example, a pressure sensor may be configured to provide an indication of the fluid pressure detected in a fluid pathway between the patient interface 186 and reservoir 124. The pressure sensor may be coupled to or integral with the actuator for inserting the patient interface 186 into the user. In an example, the controller 121 may be operable to determine a rate of drug infusion based on the indication of the fluid pressure. The rate of drug infusion may be compared to an infusion rate threshold, and the comparison result may be usable in determining an amount of insulin onboard (IOB) or a total daily insulin (TDI) amount. In one embodiment, analyte sensor 108 may be integral with drug delivery device 102.

Drug delivery device 102 further includes a power source 128, such as a battery, a piezoelectric device, an energy harvesting device, or the like, for supplying electrical power to controller 121, memory 123, drive mechanisms 125 and/or other components of drug delivery device 102.

Drug delivery device 102 may be configured to perform and execute processes required to deliver doses of the medication to the user without input from the user device 105 or the optional accessory device 106. As explained in more detail, MDA 129 may be operable, for example, to determine an amount of insulin to be delivered, IOB, insulin remaining, and the like and to cause controller 121 to activate drive mechanism 125 to deliver the medication from reservoir 124. MDA 129 may take as input data received from the analyte sensor 108 or from user app 160.

The reservoirs 124, 124-2 may be configured to store drugs, medications or therapeutic agents suitable for automated delivery, such as insulin, Pramlintide, GLP-1, co-formulations of insulin and GLP-1 or pramlintide, glucagon, morphine, blood pressure medicines, arthritis drugs, chemotherapy drugs, fertility drugs, hormonal drugs, or the like.

Drug delivery device 102 may be a wearable device and may be attached to the body of a user, such as a patient or diabetic, at an attachment location and may deliver any therapeutic agent, including any drug or medicine, such as insulin or the like, to a user at or around the attachment location. A surface of drug delivery device 102 may include an adhesive to facilitate attachment to the skin of a user.

When configured to communicate with an external device, such as the user device 105 or the analyte sensor 108, drug delivery device 102 may receive signals over the wired or wireless link 194 from the user device 105 or from the analyte sensor 108. The controller 121 of drug delivery device 102 may receive and process the signals from the respective external devices as well as implementing delivery of a drug to the user according to a diabetes treatment plan or other drug delivery regimen.

### Accessory Device

Optional accessory device 106 may be, a wearable smart device, for example, a smart watch (e.g., an Apple Watch®), smart eyeglasses, smart jewelry, a global positioning system-enabled wearable, a wearable fitness device, smart clothing, or the like. Accessory device 106 may alternatively be a smart insulin pen that works with drug delivery device 102 in managing blood glucose and treating diabetes of a user. Similar to user device 105, the accessory device 106 may also be configured to perform various functions including controlling or communicating with drug delivery device 102. For example, the accessory device 106 may include a communication interface 174, a processor 171, a user interface 178 and a memory 173. The user interface 178 may be a graphical user interface presented on a touchscreen display of the smart accessory device 107. The memory 173 may store programming code to operate different functions of the smart accessory device 107 as well as an instance of the user app 160, or a pared-down version of user app 160 with reduced functionality. In some instances, accessory device 107 may also include sensors of various types.

### Analyte Sensor

The analyte sensor 108 may include a controller 131, a memory 132, a sensing/measuring device 133, an optional user interface 137, a power source/energy harvesting circuitry 134, and a communication interface 135. The analyte sensor 108 may be communicatively coupled to the processor 151 of the management device 105 or controller 121 of drug delivery device 102. The memory 132 may be configured to store information and programming code 136.

The analyte sensor 108 may be configured to detect one or multiple different analytes, such as glucose, lactate, ketones, uric acid, sodium, potassium, alcohol levels or the like, and output results of the detections, such as measurement values or the like. The analyte sensor 108 may, in an exemplary embodiment, be configured as a continuous glucose monitor (CGM) to measure blood glucose values at a predetermined time interval, such as every 5 minutes, every 1 minute, or the like. The communication interface 135 of analyte sensor 108 may have circuitry that operates as a transceiver for communicating the measured blood glucose values to the user device 105 over a wireless link 195 or with drug delivery device 102 over the wireless communication link 108. While referred to herein as an analyte sensor 108, the sensing/measuring device 133 of the analyte sensor 108 may include one or more additional sensing elements, such as a glucose measurement element, a heart rate monitor, a pressure sensor, or the like. The controller 131 may include discrete, specialized logic and/or components, an application-specific integrated circuit, a microcontroller or processor that executes software instructions, firmware, programming instructions stored in memory (such as memory 132), or any combination thereof.

Similar to drug delivery device 102, CGM 108 may be provided with one or more light sources 140 providing red and near-infrared light to the wound site where the sensing/measuring device 133 was inserted into the skin.

Similar to the controller 121 of drug delivery device 102, the controller 131 of the analyte sensor 108 may be operable to perform many functions. For example, the controller 131 may be configured by programming code 136 to manage the collection and analysis of data detected by the sensing and measuring device 133.

Although the analyte sensor 108 is depicted in FIG. 1 as separate from drug delivery device 102, in various embodiments, the analyte sensor 108 and drug delivery device 102 may be incorporated into the same unit. That is, in various examples, the analyte sensor 108 may be a part of and integral with drug delivery device 102 and contained within the same housing as drug delivery device 102 or an attachable housing thereto. In such an example configuration, the controller 121 may be able to implement the functions required for the proper delivery of the medication alone without any external inputs from user device 105, the cloud-based services 111, another sensor (not shown), the optional accessory device 106, or the like.

### Cloud-Based Services

Drug delivery system 100 may communicate with or receive services from a cloud server 122 providing cloud-based services 111. Services provided by cloud server 112 may include data storage that stores personal or anonymized data, such as blood glucose measurement values, historical IOB and/or TDI, prior carbohydrate-compensation dosage, and other forms of data. In addition, the cloud-based services 111 may process anonymized data from multiple users to provide generalized information related to TDI, insulin sensitivity, IOB and the like. The communication link 115 that couples the cloud server 112 to other components of system 100, for example, devices 102, 105, 106, 108 of system 100 may be a cellular link, a Wi-Fi link, a Bluetooth® link, or a combination thereof.

### Communication Links

The wireless communication links 115 and 191-196 may be any type of wireless link operating using known wireless communication standards or proprietary standards. As an example, the wireless communication links 191-196 may provide communication links based on Bluetooth®, Zigbee®, Wi-Fi, a near-field communication standard, a cellular standard, or any other wireless protocol via the respective communication interfaces 126, 135, 154 and 174.

### Operational Example

In an operational example, user application 160 implements a graphical user interface that is the primary interface with the user and may be used to activate drug delivery device 102, trigger a needle/cannula insertion, start and stop drug delivery device 102, program basal and bolus calculator settings for manual mode as well as program settings specific for automated mode (hybrid closed-loop or closed-loop).

User app 160, provides a graphical user interface 158 that allows for the use of text, graphics, and on-screen instructions to prompt the user through the set-up processes and the use of system 100. It may also be used to program the user's custom basal insulin delivery profile, accept a recommended basal insulin delivery profile, check the status of drug delivery device 102, initiate bolus doses of insulin, make changes to a patient's insulin delivery profile, handle system alerts and alarms, or allow the user to switch between automated mode and manual mode.

User app 160 may be configured to operate in a manual mode in which user app 160 will deliver insulin at programmed basal rates and user-defined bolus amounts with the option to set temporary basal profiles. The controller 121 will also have the ability to function as a sensor-augmented pump in manual mode, using sensor glucose data provided by the analyte sensor 108 to populate the bolus calculator.

User app 160 may be configured to operate in an automated mode in which user app 160 supports the use of one or multiple target blood glucose values that may be adjusted manually or automatically by the system. For example, in one embodiment, target blood glucose values can range from 110-150 mg/dL, in 10 mg/dL increments, in 5 mg/dL increments, or other increments, but preferably 10 mg/dL increments. The experience for the user will reflect current setup flows whereby the healthcare provider assists the user to program basal rates, glucose targets and bolus calculator settings. These in turn will inform the user app 160 for insulin dosing parameters. The insulin dosing parameters will be adapted over time based on the total daily insulin (TDI) delivered during each use of drug delivery device 102. A temporary hypoglycemia protection mode or an activity mode may be implemented by the user for various time durations in automated mode. With hypoglycemia protection mode or an activity mode, the algorithm reduces insulin delivery and is intended for use over temporary durations when insulin sensitivity is expected to be higher, such as during exercise or fasting.

The user app 160 (or MDA 129) may provide periodic insulin micro-boluses based upon past glucose measurements and/or a predicted glucose over a prediction horizon (e.g., 60 minutes). Optimal post-prandial control may require the user to give meal boluses in the same manner as current pump therapy, but normal operation of the user app 160 will compensate for missed meal boluses and mitigate prolonged hyperglycemia. The user app 160 uses a control-to-target strategy that attempts to achieve and maintain a set target glucose value, thereby reducing the duration of prolonged hyperglycemia and hypoglycemia.

In some embodiments, user device 105 and the analyte sensor 108 may not communicate directly with one another. Instead, data (e.g., blood glucose readings) from analyte sensor may be communicated to drug delivery device 102 via link 196 and then relayed to user device 105 via link 194. In some embodiments, to enable communication between analyte sensor 108 and user device 105, the serial number or other identifier of the analyte sensor may be entered into user app 160.

User app 160 may provide the ability to calculate a suggested bolus dose through the use of a bolus calculator. The bolus calculator is provided as a convenience to the user to aid in determining the suggested bolus dose based on ingested carbohydrates, most-recent blood glucose readings (or a blood glucose reading if using fingerstick), programmable correction factor, insulin to carbohydrate ratio, target glucose value, and insulin on board (IOB). IOB is estimated by user app 160 taking into account any manual bolus and insulin delivered by the algorithm.

### Description of Embodiments

The accuracy of a CGM is often expressed in terms of a mean absolute relative difference (MARD) computed using temporally-matched glucose data from CGM systems in comparison glucose measurements obtained (most often) by capillary blood glucose measurements performed in the setting of a clinical study. A typical CGM may have a built-in compensation algorithm to compensate for the MARD over the life of the CGM. Typically, the MARD will be relatively high at the beginning of the service life of the CGM after it is initially installed due to the body's wound healing response or reaction to the introduction of the sensor under the skin of the user. Various embodiments of the invention, as described herein, are able to lower the MARD value more quickly to a level at which the compensation algorithm of the CGM is able to effectively compensate. This is accomplished by applying, in one embodiment of the invention, optical energy in the form of red and near-infrared light to the wound site.

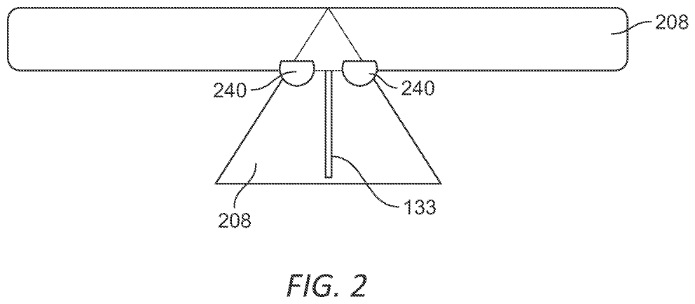

In a first embodiment of the invention, shown in FIG. 2, medical device 208 (which may be CGM 108 or drug delivery device 102) may be provided with one or more non-coherent light sources 240 (which may be light sources 140 or 139) providing red and near-infrared light to the wound site. The light sources 240 may be, for example, one or more LEDs having a peak emission wavelength in the visible red range and one or more LEDs having a peak emission wavelength in the near infrared range. It is desirable that the combination of LEDs chosen covers wavelengths in the range of 630 nm to 850 nm, such that the combination of LEDs utilized provides at least some light over the entire desired wavelength range. Although the invention is described in terms of the use of LEDs as light source 240, as would be realized, any means for emitting light in the desired wavelength range could be used as light source 240.

The LEDs or light source 240 may be arranged in any configuration in the housing of medical device 208 around and in close proximity to the location on the housing from which sensor 133 (or needle/cannula 186) extends. In embodiments wherein the sensor 133 (or needle/cannula 186) is inserted perpendicularly from the housing of medical device 208 into the skin of the user, the light source 240 may be configured to point directly downward into the skin of the user. In other embodiments wherein sensor 133 (or needle/cannula 186) is inserted into the skin of the user at an angle, the light source 240 may be placed at an angle matching or closely aligned with the angle of the sensor 133 (or needle/cannula 186).

It is desirable that the light emitted by light source 240 extend into the skin in an area directly around the location of sensor 133 (or needle/cannula 186), so as to illuminate an area shown in FIG. 2 as reference number 208, of the user's subdermal tissue within approximately 2 mm to 4 mm of the location of sensor 133 (or needle/cannula 186). FIG. 2 shows an idealized shape of the area 208 illuminated by light source 240, however, the shape of area 208 may be any shape, for example, spherical, centered around sensor 133 (or needle/cannula 186). The shape of area 208 may be affected by other factors, for example, the rate at which light is absorbed by the user's tissue.

In various embodiments, light source 240 may be illuminated under the control of programming code 136 contained in memory 132 of CGM monitor 108 and executed by controller 131 (or similar components on drug delivery device 102). Programming code 136 may cause the light source 240 to be connected to power source 134 (or 128) and to begin therapy upon insertion of sensor 133 (or needle/cannula 186) in the skin of the user. In some embodiments, therapy may be delivered to normal cells or tissue before insertion of sensor 133 (or needle/cannula 186) in a pre-conditioning mode.

In some embodiments, programming code 136 may modulate the light in various ways. For example, programming code 136 may cause the light from light source 240 to be provided in a continuous wave, in which the light is continuously delivered, or a pulsed wave mode, in which the light is quickly pulsed on and off. In addition, programming code 136 may, alternatively, or in addition to providing the light in continuous or pulsed wave modes, alternate between the one or more LEDs providing light at a first peak wavelength and the one or more LEDs providing light at a second peak wavelength. In some embodiments, programming code 136 may also vary the amplitude of the light emitted by light source 240. Any combination of illuminating the light source 240 using any combination of parameters is contemplated to be within the scope of the invention.

In various embodiments, the therapy provided by light source 240 should last at least two hours or longer after insertion of sensor 133 (or needle/cannula 186). The two-hour time period, for example, has been estimated to be the optimal time for application of the therapy based on models modeling the accuracy of the CGM based on the MARD between CGM readings from the CGM compared to a large population of users. However, as would be realized by one of ordinary skill in the art, the therapy may be provided for any period of time under control of programming code 136. After expiration of the therapy time, light source 240 may be disconnected from power source 134 (or 128) or may otherwise be unpowered and, in most cases, may remain unused for the remaining life of the CGM 108 or drug delivery device 102. It is contemplated that readings from CGM 108 will be considered unreliable or inaccurate by the automatic drug delivery system 100 during the time period when therapy is being delivered.

In an alternate embodiment of the invention, medical device 208 may provide thermal energy to the wound site. In one embodiment, sensor 133 (or needle/cannula 186) may be provided with a resistive element (not shown) powered by power source 134 (or 128) under control programming code 136 to provide thermal energy in an area of the user's tissue directly adjacent sensor 133 (or needle/cannula 186). In some embodiments, optical energy and thermal energy may be applied simultaneously or in any combination under control programming code 136, as described above.

Light source 240 can be powered by power source 134 of CGM 108 or power source 128 of drug delivery device 102 or, alternatively, by an exemplary power source that is piggybacked onto the housing of CGM 108 or drug delivery device 102 and removed after the period of therapy. In yet another alternative embodiment of the invention the drug delivery device 102 and CGM 108 may be co-located in a single housing. In this embodiment, light source 240 may be located in the housing of drug delivery device 102 and may be powered by power source 128 of drug delivery device 102.

As would be realized by one of skill in the art, many variations on the embodiments disclosed herein are possible. In particular, various light sources and methods of modulating the light sources disclosed herein are contemplated to be the within the scope of the invention and the invention is not meant to be limited by the specific embodiments disclosed herein.

To those skilled in the art to which the invention relates, many modifications and adaptations of the invention may be realized. Implementations provided herein, including sizes, shapes, ratings, compositions and specifications of various components or arrangements of components, and descriptions of specific manufacturing processes, should be considered exemplary only and are not meant to limit the invention in any way. As one of skill in the art would realize, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the scope of the invention. Accordingly, the method and apparatus disclosed herein are not to be taken as limitations on the invention but as an illustration thereof. The scope of the invention is defined by the claims which follow.

1. A continuous glucose monitor comprising:
a housing;
a sensor configured to extend through an opening of the housing and penetrate skin of a user;
one or more sources of optical energy arranged in close proximity to the opening of the housing; and
a processor executing programming code to control modulation of the one or more sources of optical energy to provide light therapy to the skin of the user.

2. The continuous glucose monitor of claim 1, wherein the one or more sources of optical energy comprise:
one or more LEDs having a peak wavelength in the visible red light range; and
one or more LEDs having a peak wavelength in the near-infrared range.

3. The continuous glucose monitor of claim 1, wherein the LEDs are arranged at an angle closely aligned with an angle from which the sensor extends from the housing.

4. The continuous glucose monitor of claim 1, wherein the one or more sources of optical energy provide the light therapy for a predetermined period of time.

5. The continuous glucose monitor of claim 4, wherein the programming code controls the one or more sources of optical energy to provide a continuous wave mode of operation.

6. The continuous glucose monitor of claim 4, wherein the programming code controls the one or more sources of optical energy to provide a pulsed wave mode of operation.

7. The continuous glucose monitor of claim 4, wherein the programming code controls the one or more sources of optical energy so as to alternate between the one or more LEDs in the red wavelength range and the one or more LEDs in the near-infrared wavelength range.

8. The continuous glucose monitor of claim 4, wherein the programming code varies the amplitude of the light emitted from the one or more sources of light.

9. The continuous glucose monitor of claim 4, wherein the predetermined period of time is approximately one to two hours.

10. The continuous glucose monitor of claim 4, wherein the predetermined period of time begins when the sensor is extended from the housing of the device and into the skin of the user.

11. The continuous glucose monitor of claim 4, wherein the predetermined period of time begins prior to the time when the sensor is extended from the housing of the device and into the skin of the user.

12. The continuous glucose monitor of claim 1 further comprising:
a resistive element under control of the programming code and co-located with the sensor to provide thermal energy around the sensor.

13. The continuous glucose monitor of claim 12, wherein the thermal energy and optical energy are provided simultaneously.

14. A method for delivering optical therapy from a wearable medical device, the method comprising:
providing a medical device comprising:
a processor;
a housing;
a sensor or cannula configured to extend through an opening of the housing and extend below a skin surface of a user;
one or more sources of optical energy arranged proximal to where the sensor or cannula extends below the skin surface of the user; and
controlling, using the processor executing programming code, modulation of the one or more sources of optical energy so as to deliver the optical energy into the skin of the user.

15. The method of claim 14, wherein the one or more sources of optical energy emit light having wavelengths in the visible red light range and in the near-infrared range.

16. The device of claim 14, wherein the one or more sources of optical energy are controlled so as to provide light therapy to an area of the skin of the user for a predetermined period of time.

17. The method of claim 14, wherein the one or more sources of optical energy are controlled so as to provide a continuous wave mode of operation.

18. The method of claim 14, wherein the one or more sources of optical energy are controlled so as to provide a pulsed wave mode of operation.

19. The method of claim 14, wherein the one or more sources of optical energy are controlled so as to alternate between one or more sources of optical energy emitting light in the red wavelength range and one or more sources of optical energy emitting light in the near-infrared wavelength range.

20. The method of claim 14, wherein the one or more sources of optical energy are controlled so as to vary the amplitude of the light emitted.

21. The method of claim 14, wherein the medical device further comprises a resistive element, and the method further comprises controlling the resistive element to provide thermal energy.
